(19) United States
(12) Patent Application Publication
(10) Pub. No.: US 2006/0281152 A1
(43) Pub. Date: Dec. 14, 2006

(54) Glycosaminoglycans derived from the K5 polysaccharide having high anticoagulant and antithrombotic activity and process for their preparation

(76) Inventors: Giorgio Zoppetti, Milan (IT); Pasqua Oreste, Milan (IT); Giovanni Cipolletti, Milan (IT)

Correspondence Address: ABELMAN, FRAYNE & SCHWAB;10TH FLOOR, 666 THIRD AVENUE, NEW YORK, NY, 10017, US

(21) Appl. No.: 11/440,749

(22) Filed: May 24, 2006

Related U.S. Application Data
(63) Continuation of application No. 10/240,606, filed on Dec. 21, 2002.

(30) Foreign Application Priority Data
Mar. 30, 2000 (IT) MI 2000 A 000 665

(51) Int. Cl. C12P 19/28; C08B 37/00

(52) U.S. Cl. 435/85; 536/54

(57) ABSTRACT

Glycosaminoglycans derived from the K5 polysaccharide having high anticoagulant and antithrombotic activity obtained by a process comprising the preparation of the K5 polysaccharide from Escherichia coli, N-deacetilation/N-sulfation, C-5 epimerization, supersulfation, selective O-desulfation, selective 6-O sulfation and N-sulfation, wherein said epimerization is carried out using the glucuronosyl C-5 epimerase enzyme in solution or in immobilized form in presence of specific divalent cations.

### PRIOR ART

The glycosaminoglycans are biopolymers industrially extracted from different animal organs such as the intestinal mucosa, the lung etc. According to their structure, the glycosaminoglycans are divided in heparin, heparan sulfate, dermatan sulfate, chondroitin sulfate and ialuronic acid. In particular heparin and heparan sulfate are composed of repeating disaccharide units consisting of an uronic acid (L-iduronic or D-glucuronic) and an amino sugar (glucosamine).

The uronic acid may be sulfate in position 2 and the glucosamine may be mostly N-acetilated (heparan sulfate) or N-sulfate (heparin) and 6-O sulfate. Moreover the glucosamine may also contain a sulfate group in position 3.

Heparin and heparan sulfate are polydispersed molecules having a molecular weight ranging from 3,000 to 30,000 D.

Beside the known anticoagulant and antithrombotic activity, to heparin an antilipemic, antiproliferative, antiviral, antitumor and antiangiogenetic activity is also recognized. In order to satisfy the greater request of raw material for these new therapeutic areas a new productive way alternative to the extraction from animal tissues is needed. The natural biosynthesis of heparin in mammalians and its properties have been described by Lindhal et al., 1986 in Lane D. and Lindahl U. (Eds.) “Heparin-Chemical and Biological Properties; Clinical Applications”, Edward Arnold, London, pp. 159-190 and Lindahl U. Feingold D. S. and Rodén L., (1986) TIBS, 11, 221-225.

Fundamental for the heparin activity is the sequence consisting of the pentasaccharide region bonding for the antithrombin III (ATIII), called active pentasaccharide, which is the structure needed for the high affinity bond of heparin for ATII. This sequence contains the only unit of glucosamine sulfate in position 3, which is not present in the other parts of the heparin chain. Beside the activity through ATIII, heparin exerts the anticoagulant and antithrombotic activity activating the heparin cofactor 11 (HCII) with a subsequent selective inhibition of thrombin. It is known that the minimum saccharide sequence needed to activate HCII is a chain containing at least 24 monosaccharides (Tollefsen D. M., Seminars in Thrombosis and Hemostasis 16,66-70 (1990)).

From previous studies it is known that the K5 capsular polysaccharide isolated from the Escherichia Coli strain described by Vann W. F., Schmidt M. A., Jann B., Jann K. (1981) in Eur. J. Biochem 116,359-364 shows the same sequence of the precursor of heparin and heparan sulfate (N-acetyl heparosan). This compound has been chemically modified as described by Lormeau et al. in the U.S. Pat. No. 5,550,116 and by Casu et al. (Carb. Res 263-1994-271-284) or chemically and enzymatically as described by Jann et al. (WO 92/17509) and by Casu et al., Carb. Letters 1,107-114 (1994). These modifications result in products having biological activities in the in vitro tests about coagulation that however are not at the level of heparin from extraction from animal organs.

### SUMMARY

We have found new glycosaminoglycans derived from the K5 polysaccharide from Escherichia coli, having molecular weight from 2,000 to 30,000, containing from 25 to 50% by weight of the chains having high affinity for ATIII and having a high anticoagulant and antithrombotic activity which expressed as a ratio between the HCII/antiXa activities, lies in the range from 1.5 to 4, with a prevalence of the activities implicating the inhibition of thrombin.

Said glycosaminoglycans are prepared by a process comprising several steps of chemical and enzymatic treatment and characterized by a D-glucuronic acid to L-iduronic acid epimerization step using the glucuronosyl C-5 epimerase enzyme in solution or in immobilized form in presence of specific divalent cations, said enzyme being selected from the group consisting of recombinant glucuronosyl C-5 epimerase, glucuronosyl C-5 epimerase from murine mastocytoma and glucuronosyl C-5 epimerase from extraction from cattle-liver and said divalent cations being selected from the group consisting of Ba, Ca, Mg and Mn.

### DETAILED DESCRIPTION OF THE INVENTION

The present invention refers to the glycosaminoglycans derived from the K5 polysaccharide from Escherichia coli (below also simply called K5), obtained by a process comprising the following steps:
a) Preparation of the K5 polysaccharide from Escherichia coli
b) N-deacetilation/N-sulfation
c) C-5 epimerization
d) Supersulfation
e) Selective 0-desulfation
f) (Optional) selective 6-0-sulfation
g) N-sulfation

The various steps of the process are described in detail as follows.

a) Preparation of the K5 Polysaccharide from Escherichia coli

A fermentation in an Erlenmeyer flask is first carried out according to the M199A001465 patent and using the following medium:

| | | |
|---|---|---|
| Degreased soy flour | 2 | gr/l |
| K₂HPO₄ | 9.7 | gr/l |
| KH₂PO₄ | 2 | gr/l |
| MgCI₂ | 0.11 | gr/l |
| Sodium citrate | 0.5 | gr/l |
| Ammonium sulfate | 1 | gr/l |
| Glucose | 2 | gr/l |
| Spring water | 1,000 | ml |
| pH = 7.3 | | |

The medium is sterilized at 120° C. for 20 minutes.

The glucose is separately prepared in form of solution which is sterilized at 120° C. for 30 minutes and added to the medium in a sterile way.

The Erlenmeyer flask is inoculated with a suspension of E. coli Bi 8337/41 cells (010:K5:H4) coming from a slant kept in Triptic soy agar, and incubated at 37° C. for 24 hours under controlled stirring (160 rpm, 6 cm run). The bacterial growth is measured counting the cells with the microscope.

In a subsequent operation, a 14 1 Chemap-Braun fermenter containing the same previously mentioned medium, is inoculated at 0.1% with the culture of the above Erlenmeyer flask and the fermentation is carried out by aeration of 1 vvm,=air volume per liquid volume per minute), 400 rpm stirring and 37° C. temperature for 18 hours.

During the fermentation pH, oxygen, the residual glucose, the produced K5 polysaccharide and the bacterial growth are measured.

At the end of the fermentation the temperature is taken to 80° C. for 10 minutes.

The cells are separated from the medium by 10,000 rpm centrifugation and the supernatant is ultrafiltered using a SS 316 module (MST) provided with PES membranes having 800 and 10,000 D nominal cut-off to reduce the volume to ⅕.

The K5 polysaccharide is then precipitated by addition of 4 volumes of acetone at 4° C. and allowed to sedimentate overnight at 4° C., and finally it is recovered by 10,000 rpm centrifugation for 20 minutes or filtration.

Then the deproteinization of the obtained solid is carried out using a type 11 protease from Aspergillus Orizae in 0.1 M NaCI buffer and 0.15 M EDTA at pH 8 containing 0.5% SDS (10 mg/l filtrate) at 37° C. for 90 minutes.

The obtained solution is ultrafiltered on SS 316 module with membranes having 10,000 D nominal cut-off with 2 extractions with 1 M NaCI and washed with water to absorbance disappearance in the ultrafiltrate. The K5 polysaccharide is then precipitated with acetone and a 850 mg per liter of fermenter yield is obtained.

The purity of the obtained polysaccharide is measured by the determination of the uronic acids (carbazole method), proton and carbon 13 NMR, UV and protein content. Purity turns out to be greater than 80%.

The obtained polysaccharide consists of two fractions having different average molecular weight, 30,000 and 5,000 D respectively as it results from the HPLC determination with two Bio-sil SEC 250 (Bio Rad) series columns and Na2SO4 as mobile phase at room temperature and 0.5 ml/minute flux. The measure is carried out against a standard curve obtained with known molecular weight heparin fractions.

Triton X-100 is added to a 1% aqueous solution of the purified K5 polysaccharide until the achievement of a 5% solution. It is left for 2 hours at 55° C. under stirring.

The temperature is increased to 75° C. and during the subsequent cooling at room temperature two phases are formed.

On the upper phase (organic phase) the thermal treatment is repeated with formation of the two phases, for other two times. The aqueous phase containing the polysaccharide is finally concentrated under reduced pressure and precipitated with acetone or ethanol. The organic phase is discarded. The sample purity is controlled by proton NMR and turns out to be 95%.

The yield of this treatment turns out to be 90%. b) N-deacetilation/N-sulfation 10 g of purified K5 polysaccharide are solubilized in 100-2,000 ml of 2N sodium hydroxide and left to react at 40-80° C. for the time needed for the complete deacetylation, which is never greater than 30 hours. The solution is taken to room temperature and to neutral pH with 6N hydrochloric acid.

The solution containing the deacetilated K5 is maintained at 20-65° C. and added with 10-40 g of sodium carbonate with single addition and with 10-40 g of a sulfating agent selected among the available reagents such as the pyridine- sulfotrioxide adduct, trimethylamine-sulfotrioxide etc.

The addition of the sulfating agent is carried out in a variable time to 12 hours. At the end of the reaction, if necessary, the solution is taken to room temperature, then to pH 7.5-8 with a 5% hydrochloric acid solution.

The product is purified from the salts by known techniques such as for example by diafiltration using a 1,000 D spiral membrane (prepscale cartridge-Millipore). The process is ended when the permeate conductivity is lower than 1,000 pS, preferably lower than 100 pS. The obtained product is reduced in volume until the achievement of a 10% polysaccharide concentration using the same filtering system in concentration. The concentrated solution, if necessary, is dried by common methodologies.

The N-sulfate/N-acetyl ratio turns out to be from 10/0 to 7/3 measured by carbon 13 NMR. c) C-5 epimerization: The C-5 epimerization step according to the present invention may be carried out by glucuronyl C-5 epimerase enzyme (also simply called C-5 epimerase) in solution or in immobilized form.

C-5 epimerization with in solution enzyme From 1.2×10′ to 1.2×10″ cpm (counts per minute) of natural or recombinant C-5 epimerase enzyme, computed according to the method described by Campbell P. et al., Analytical Biochemistry 131,146-152 (1983), are dissolved in 2-2,000 mi of 25 mM Hepes buffer at a pH from 5.5 to 7.4, containing 0.001-10 g of N-deacetilated N-sulfate K5, and one or more ions selected among barium, calcium, magnesium, manganese at a concentration between 10 and 60 mM. The reaction is carried out at a temperature between 30 and 40° C., preferably 37° C., for 1-24 hours. At the end of the reaction the enzyme is inactivated at 100° C. for 10 minutes.

The product is purified by passage on DEAE resin or DEAE Sartobind cartridge and removed by 2M NaCI and finally desalted on Sephadex G-10 resin or it is purified by precipitation with 2 ethanol volumes and passage on IR 120 H+ resin to retransform it in sodium salt.

A product having an iduronic acid/glucuronic acid ratio ranging from 40:60 to 60:40 computed by ′H-NMR as already described in the W096/14425 patent is obtained.

C-5 Epimerization with Immobilized Enzyme

The C-5 epimerase enzyme, natural or recombinant, may be immobilized on various inert supports which may be resins or membranes or glass beads derivatized with reactive functional groups using the common bond techniques for the enzymes for example by cyanogen bromide, by glutaraldehyde, by carbodiimide or by reacting the enzyme with a ionic exchange resin or making it to be adsorbed on a membrane. According to the present invention, the attack reactions of the enzyme to the inert support are carried out in the presence of the N-deacetilated N-sulfate K5 substrate in order to avoid that the bond occurs through the active site of the enzyme with subsequent activity loss.

The measurement of the immobilized enzyme activity is carried out by recirculating through a column containing the immobilized enzyme the amount of N-deacetilated N-sulfate K5 theoretically convertible by the cpm of immobilized enzyme, dissolved in 25 mM Hepes buffer, 0.1 M KCI, 0.01% Triton X100 and 0.15 M EDTA at pH 7.4 at 37° C. overnight with 0.5 ml/minute flux. After the purification by DEAE chromatographic system and desalting on Sephadex G-10 the product is freeze-dried and tested for the iduronic acid content by the proton NMR technique.

The iduronic acid/glucuronic acid ratio must be about 30:70. 20-1,000 ml of a 25 mM Hepes solution at pH between 6 and 7.4 containing one or more ions selected among barium, calcium, magnesium, manganese in a concentration ranging from 10 to 60 mM and 0.001-10 g of N-deacetilated N-sulfate K5, thermostated at a temperature between 30 and 40° C., are recirculated at a 30-160 ml/h flux for a time ranging from 1 to 24 hours in a column containing from 1.2×107 to 3×10′ cpm equivalents of the immobilized enzyme on the inert support thermostated at a temperature ranging from 30 to 40° C. At the end of the reaction the sample is purified by the same procedures pointed out in the epimerization in solution.

The obtained product exhibits a ratio between iduronic acid and glucuronic acid ranging from 40:60 to 60:40.

d) Supersulfation

The solution containing the epimerized product of the step c) at a 10% concentration is cooled to 10° C. and then passed through IR-120 H+ cationic exchange resin or equivalent (35-100 ml). Both the column and the container of the eluate are maintained at 10° C. After the passage of the solution the resin is washed with deionized water until the permeate pH is greater than 6 (about 3 volumes of deionized water). The acid solution is taken to neutrality with a tertiary or quaternary amine such as for example tetrabutylammonium hydroxide (15% aqueous solution) obtaining the relative ammonium salt. The solution is concentrated at minimum volume and freeze-dried. The obtained product is suspended in 20-500 ml of DMF or DMSO and added with 15-300 g of a sulfating agent such as the pyridine-S03 adduct in solid form or in a solution of DMF or DMSO. The solution is maintained at 20-70° C., preferably between 40-60° C. for 2-24 hours.

At the end of the reaction the solution is cooled to room temperature and added with acetone saturated with sodium chloride to the complete precipitation.

The precipitate is separated from the solvent by filtration, solubilized with the minimum amount of deionized water (for example 100 ml) and added with sodium chloride until the achievement of a 0.2 M solution. The solution is taken to pH 7.5-8 with 2N sodium hydroxide and added with acetone until complete precipitation.

The precipitate is separated from the solvent by filtration. The obtained solid is solubilized with 100 ml of deionized water and purified from the residual salts by ultrafiltration as described in step b).

An aliquot is freeze-dried for the structural analysis of the supersulfated product by ¹³C-NMR.

The obtained product turns out to have a sulphates per disaccharide content equal to 2.0-3.5 computed according to Casu et al., Carbohyd. Res. Vol. 39, pp 168-176 (1975). The position 6 of the aminosugar is 80÷95% sulfated and the position 2 is fully not sulfate.

The other sulfate groups are present in the position 3 of the aminosugar and 2 and 3 of the uronic acid.

e) Selective O-desulfation

The solution containing the product obtained from step d) is passed through IR-120 H⁺ cationic exchange or equivalent (35-100 ml). After the passage of the solution the resin is washed with deionized water until the pH of the permeate is greater than 6 (about 3 volumes of deionized water). The acid solution is taken to neutrality by pyridine addition.

The solution is concentrated to minimum volume and freeze-dried. The obtained product is treated with 20-2,000 ml of a DMSO/methanol (9/1 V/V) solution and the obtained solution is kept at 45-90° C. for 1-8 hours. At the end the solution is added with 10-200 ml of deionized water and then it is treated with acetone saturated with sodium chloride in an amount such as to complete the precipitation.

With the selective O-desulfation first the sulfate groups are removed from the position 6 of the aminosugar, then those ones of the positions 3 and 2 of the uronic acid and finally that one of the position 3 of the aminosugar.

The obtained solid is purified by diafiltration as described in step b).

An aliquot is freeze-dried for the structural analysis by ¹³C-NMR.

In case the NMR analysis reveals a content of sulphates in position 6 of the aminosugar greater than 60%, computed as described by Casu et al. Arzneimittel- forschiung Drug Research 33-1,135-142 (1983) one goes directly to step g).

Otherwise one goes on with the following step.

f) Selective 6-O-sulfation (Optional)

The solution containing the product of the step e) is treated as described in step d) to obtain the tertiary or quaternary salt, operating however at 20-25° C.

The ammonium salt is suspended in 20-500 ml of DMF. The suspension is cooled to 0° C. and treated with an amount of a sulfating agent such as the pyridine-S0₃ adduct computed as a function of the percentage of sulfate in position 6 of the aminosugar to be restored considering a minimum of 60% of 6-O sulfate computed as described above. Such an amount of sulfating agent is between two and ten equivalents with respect to the hydroxyl functions to sulfate. The sulfating agent is added by single addition or with subsequent additions in a maximum total time of 20 minutes.

The sulfating agent may be in powder or dissolved in a little amount of DMF.

The solution is kept to 0-5° C. for 0.5-3 hours. The solution is then treated with acetone saturated with sodium chloride in amounts such to complete the precipitation.

The obtained solid is purified by diafiltration as described in step b).

An aliquot is freeze-dried for the structural analysis by ¹³C-NMR.

In case the 6-0-sulfate content is lower than 60% as measured by NMR technique, the step f) is repeated.

g) N-sulfation

The solution coming from the step f) or, possibly, from step e) is treated as described in step b) for the N-sulfation.

The glycosaminoglycans obtained by the process of the invention are characterized by proton and carbon 13 NMR and by biological tests such as antiXa, APTT, HCII, Anti lla and affinity for ATIII.

The obtained product may be submitted to fractioning by column chromatographic technique or by ultrafiltration obtaining fractions having low molecular weight from 2,000 to 8,000 D and high molecular weight from 25,000 to 30,000 D or the product may be submitted to depolymerization controlled by known techniques such as for example the deamination with nitrous acid as described in W08203627.

The typical characteristics concerning the biological activity of the glycosaminoglycans obtained by the process of the invention (IN-2018 UF and IN-2018 LMW) are reported in Table 1, in comparison with Heparin UF (4^{th} Int. Standard) and LMW Heparin (1^{st} int. Standard).

TABLE 1
Biological Activity of the product obtained by the described process

| | UF Heparin | LMW Heparin | | |
|---|---|---|---|---|
| | (4^{th} Int. | (1^{st} Int. | IN-2018 | IN-2018 |
| Sample | Standard | Standard | UF | LMW |
| 1-Anti Xa | 100 | 84 | 70-250 | 40-100 |
| 2-APTT | 100 | 30 | 40-90 | 25-80 |
| 3-HCII | 100 | n.d. | 300-500 | 100-200 |
| 4-Anti IIa | 100 | 33 | 100-600 | 20-210 |
| 5-Average | 13500 | 4500 | 18000- | 4000- |
| Molecular | | | 30000 | 8000 |
| Weight | | | | |
| 6-ATIII | 35% | n.d. | 25-50 | 20-40 |
| Affinity | | | | |

### REFERENCES

1. Thomas D. P. et al., Thrombosis and Haemostasis 45, 214-(1981) against the IV heparin international standard.
2. Anderson et al., Thrombosis Res. 9,575 (1976) against the IV heparin international standard.
3. The test is carried out mixing 20 ml of HCII (Stago) 0.05 PEU/ml dissolved in water with 80 ul of a solution of the sample under examination at different concentrations and 50 pl of thrombin (0.18 U/ml-Boehringer) in 0.02 M tris buffer, pH 7.4, containing 0.15 M NaCI and 0.1% PEG-6000. The solution is incubated for 60 sec. at 37° C., then 50 pl of 1 mM Spectrozyme (American Diagnostic) chromogenic substrate are added. The reaction is recorded in continuum for 180 sec. with readings every second at 405 nm using a ACL-7000 (IL) automatic coagulometer.
4. The test is carried out mixing 30 pi of a 0.5 U/ml ATIII (Chromogenix) solution dissolved in 0.1 M tris buffer, pH 7.4, with 30 ul of a solution of the sample under examination at different concentrations and 60 pl of thrombin (5.3 nKat/ml-Chromogenix) in 0.1 M pH 7.4 tris buffer. The solution is incubated for 70 sec. at 37° C., then 60 pl of 0.5 mM S-2238 (Chromogenix) chromogenic substrate in water are added.

The reaction is recorded in continuum for 90 sec. with readings each second at 405 nm using a ACL-7000 (IL) automatic coagulometer.
5. Harenberg and De Vries, J. Chromatography 261, 287-292 (1983) 6. Hook M. et al. FEBS Letters 66,90-93 (1976).

From the Table it is pointed out that the product obtained with the present process shows an activity comparable with the extractive heparin in the test referred to the Xa (1) factor and reduced the global (2) anticoagulant activity while the values of the test referring to the inhibition of thrombin (3,4) turn out to be significantly greater. These characteristics configure in the obtained product greater antithrombotic properties and less side effects such as the bleeding effect with respect to the extractive heparin.

Thanks to their characteristics, the glycosaminoglycans according to the present invention may be used, alone or in form of combinations with pharmaceutically acceptable excipients or diluents, for the anticoagulant and antithrombotic treatment.

Therefore the present invention also includes the compositions containing an effective amount of said glycosaminoglycans in combination with pharmaceutically acceptable excipients or diluents.

Finally the present invention also refers to a therapeutic method including the administration of an effective amount of said glycosaminoglycans for the anticoagulant and antithrombotic treatment.

The following Examples are reported for illustrative aim of the invention.

### EXAMPLE 1

The Example 1 is carried out according to the following steps:

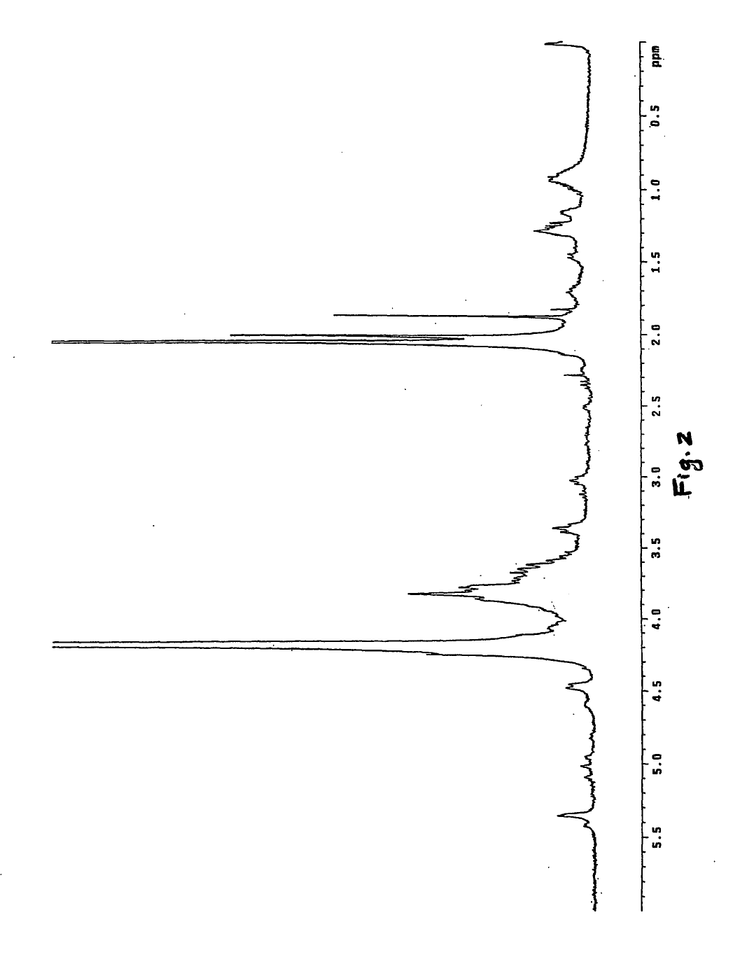

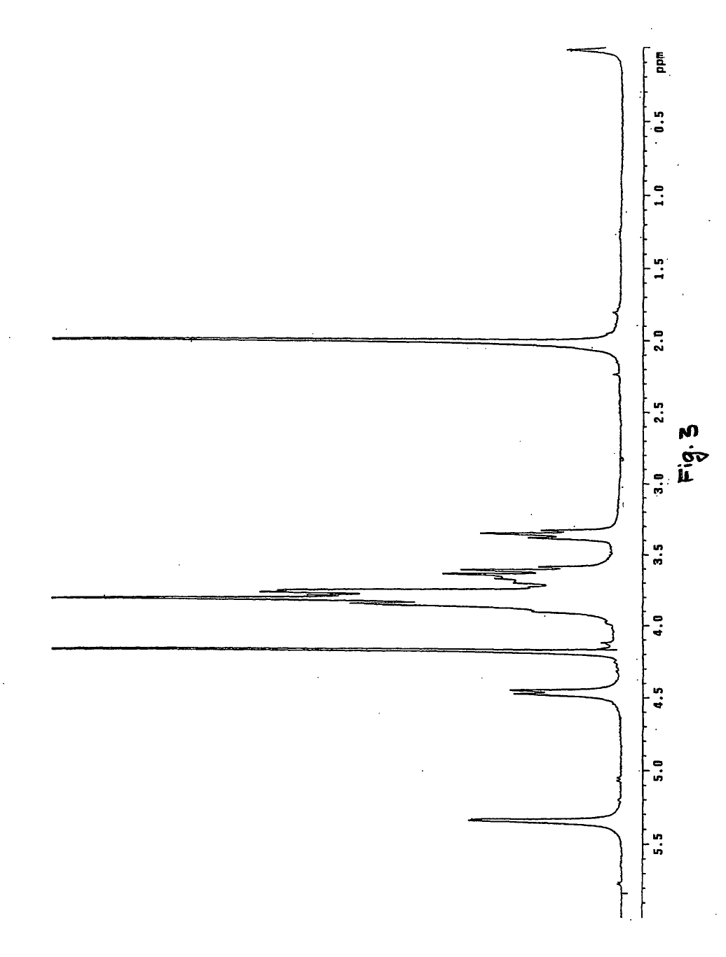

a) 10 g. of the K5 polysaccharide obtained by fermentation as described in the M199A001465 patent having 80% purity (FIG. 2) are dissolved in deionized water in order to obtain a 1% solution. Triton X-100 is added to obtain a 5% solution and the solution is kept for 2 hours at 55° C. under stirring.

The solution is heated to 75° C. and kept at this temperature until the formation of an homogeneous turbid system and then the solution is quickly cooled to room temperature.

In the cooling two phases are formed.

On the upper phase (organic phase) said thermal treatment is repeated for other two times. The aqueous phase containing the K5 polysaccharide is finally concentrated to 1/10 of the volume under reduced pressure and precipitated with acetone or ethanol.

The organic phase is discarded.

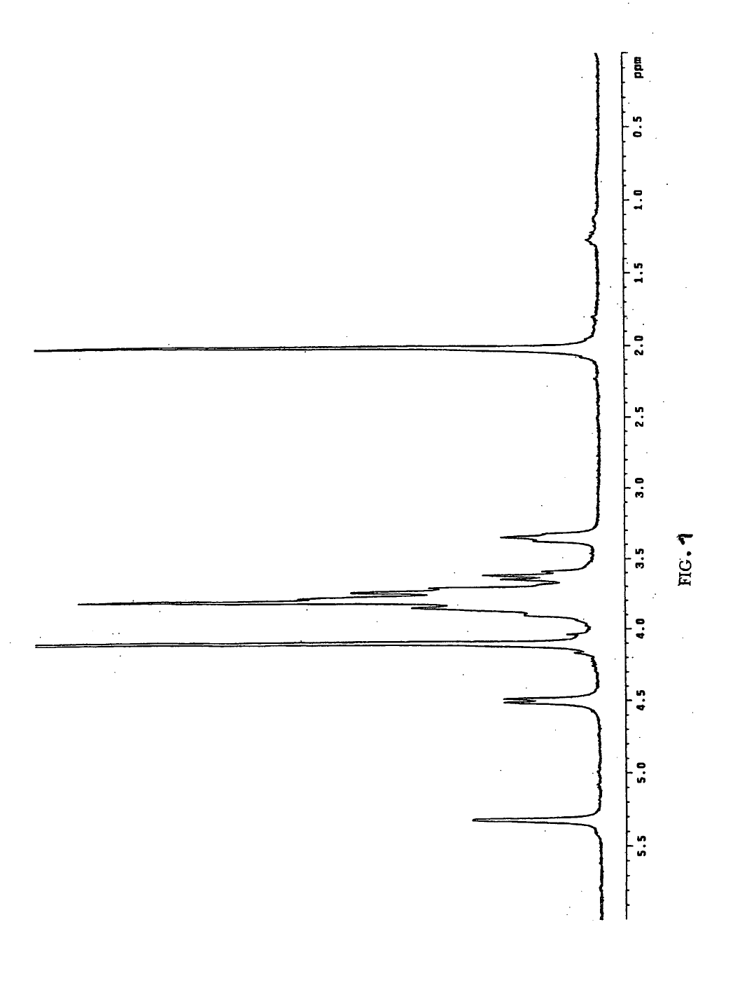

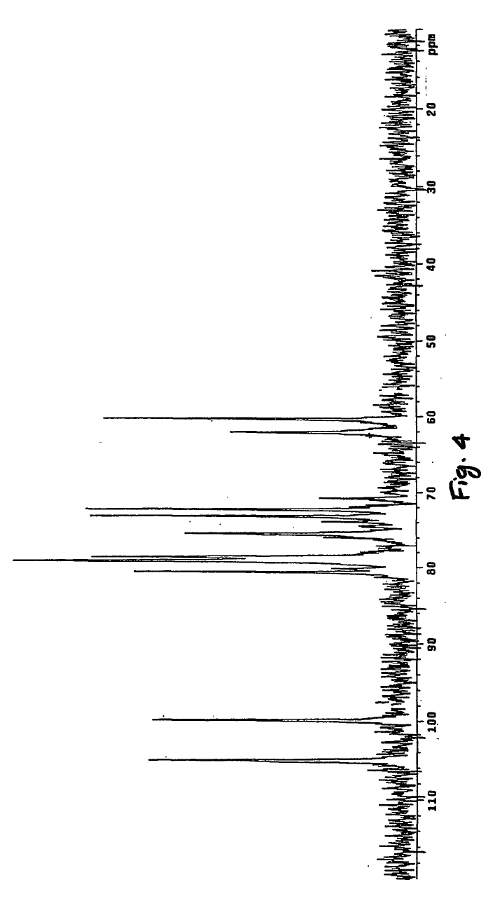

The recovered product consists of 90% purity K5 polysaccharide, controlled by proton NMR (FIG. 3) with respect to the spectrum of the internal standard (FIG. 1).

b) The product obtained from step a) is solubilized with 1,000 mi of 2N sodium hydroxide and left at 60° C. for 18 hours. The solution is taken to room temperature and then to neutral pH with 6N hydrochloric acid. One thus obtains the N-deacetilated K5 polysaccharide.

The solution containing the N-deacetilated K5 is maintained at 40° C. and added with 10 g of sodium carbonate with single addition and 10 g. of pyridine-sulfotrioxide adduct in 10 minutes. At the end of the reaction, the solution is taken to room temperature, then to pH 7.5-8 with a 5% hydrochloric acid solution.

The obtained product, consisting of the N-deacetilated N-sulfate K5 polysaccharide, is purified from the salts by diafiltration using a 1,000 D (prepscale cartridge-Millipore) spiral membrane. The purification process is ended when the permeate conductivity is lower than 100 μS.

The product kept by the membrane is taken to a 10% polysaccharide concentration using the same diafiltration system and then it is freeze-dried.

The N-sulfate/N-acetyl ratio in the obtained product turns out to be 9.5/0.5, measured by carbon 13 NMR (FIG. 4).

c) 1-Preparation of the immobilized C-5 epimerase enzyme

To 5 mg of recombinant C-5 epimerase obtained according to the W098/48006 patent corresponding to 1.2×10¹¹ pm (counts per minute) dissolved in 200 ml of 0.25 M Hepes buffer, pH 7.4, containing 0.1 M KCl, 0.1% Triton X-100 and 15 mM EDTA, 100 mg of N-deacetilated N-sulfate K5 are added obtained as described in step b). The solution is diafiltered in a 30,000 D membrane at 4° C. until the disappearance of the N-deacetilated N-sulfate K5 in the diafiltered. To the solution kept by the membrane is then changed the buffer by diafiltration substituting it with 200 mM NaHCO₃ at pH 7 and, after concentration at 50 ml, 50 ml of CNBr Sepharose 4b activated resin are added and it is left to react overnight at 4° C.

At the end of the reaction the amount of residual enzyme in the supernatant is measured by the Quantigold (Diversified Biotec) method after centrifugation. The enzyme in the supernatant turns out to be absent, showing that with the described method the enzyme is 100% immobilized. In order to occupy the sites of the resin remained available the resin is washed with 100 mM TRIS-HCI buffer at pH 8.

For the measurement of the activity of the immobilized enzyme, an amount of immobilized enzyme theoretically corresponding to 1.2×10⁷ cpm, is loaded into a column. In the so prepared column 1 mg of N-deacetilated N-sulfate K5 obtained as described in step b) dissolved in 25 mM Hepes buffer, 0.1 M KCl, 0.015 M EDTA, 0.01% Triton X-100, at pH 7.4, is treated making it to recirculate through said column at 37° C overnight with a 0.5 ml/minute flux.

After the purification by DEAE chromatographic system and desalting on Sephadex G10 the sample is freeze-dried and tested for the iduronic acid content by proton NMR technique as already described in the W096/14425 patent.

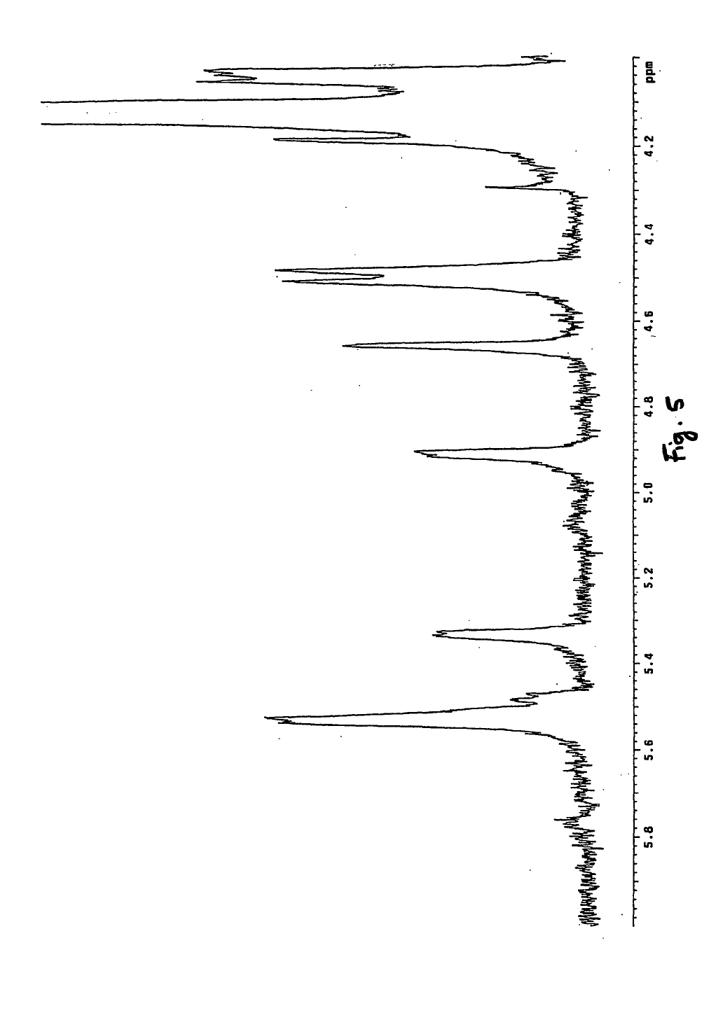

The iduronic acid/glucuronic acid ratio is 30/70. (FIG. 5).

2-Epimerization

10 g of the N-deacetilated N-sulfated K5 polysaccharide are dissolved in 600 ml of 25 mM HEPES buffer, pH 6.5, containing 50 mM Cacl₂. The obtained solution is made to recirculate through a 50 ml column loaded with the resin containing the immobilized enzyme.

This operation is carried out at 37° C. with a 200 ml/h flux for 24 hours.

The obtained product is purified by ultrafiltration and precipitation with ethanol.

The precipitate is resolubilized in water at a 10% concentration.

One obtains an epimerized product with a iduronic acid/glucuronic acid ratio equal to 48/52 against a 0/100 ratio of the starting product.

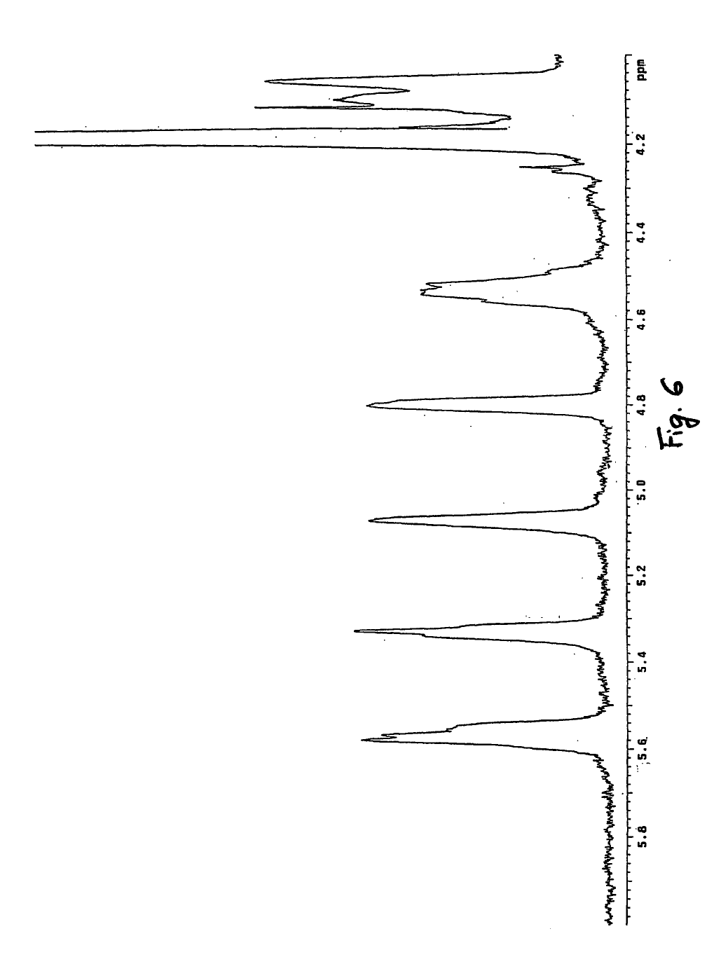

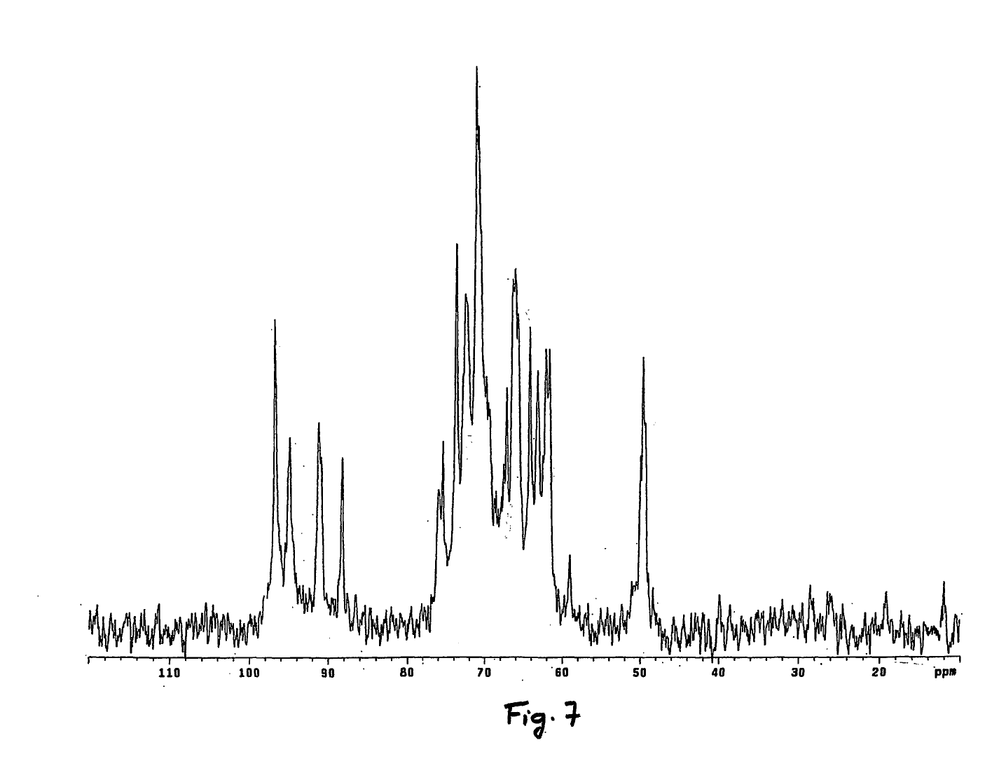

The epimerization percentage has been computed with ¹H-NMR (FIG. 6).

The yield, computed measuring the uronic acids content against standard by the carbazole method (Bitter and Muir Anal. Biochem. 39,88-92-1971) is equal to 90%.

d) The solution containing the epimerized product with 10% concentration coming from the step c) is taken to 10° C. with cooled bath and then it is passed on IR-120 H⁺ (50 ml) cationic exchange resin. Both the column and the eluate container are kept at 10° C. After the passage of the solution the resin is washed with 3 volumes of deionized water. The permeate pH turns out to be greater than 6. The acid solution is taken to neutrality with a 15% tetrabutylammonium hydroxide aqueous solution. The resulting solution is concentrated at 1/10 of the volume in a rotating evaporator at 40° C. under vacuum, and freeze-dried.

The product is suspended in 200 ml of DMF and added with 150 g of the pyridine- S0₃ adduct dissolved in 200 ml of DMF. The solution is kept at 45° C. for 18 hours.

At the end of the reaction the solution is cooled to room temperature and added with 1,200 ml of acetone saturated with sodium chloride.

The obtained precipitate is separated from the solvent by filtration, solubilized with 100 ml of deionized water and added with sodium chloride until the achievement of a 0.2 M solution. The solution is taken to pH 7.5-8 with 2 N sodium hydroxide and added with 300 ml of acetone. The precipitate is separated by filtration. The obtained solid is solubilized with 100 ml of deionized water and purified from the residual salts by diafiltration as described in step b).

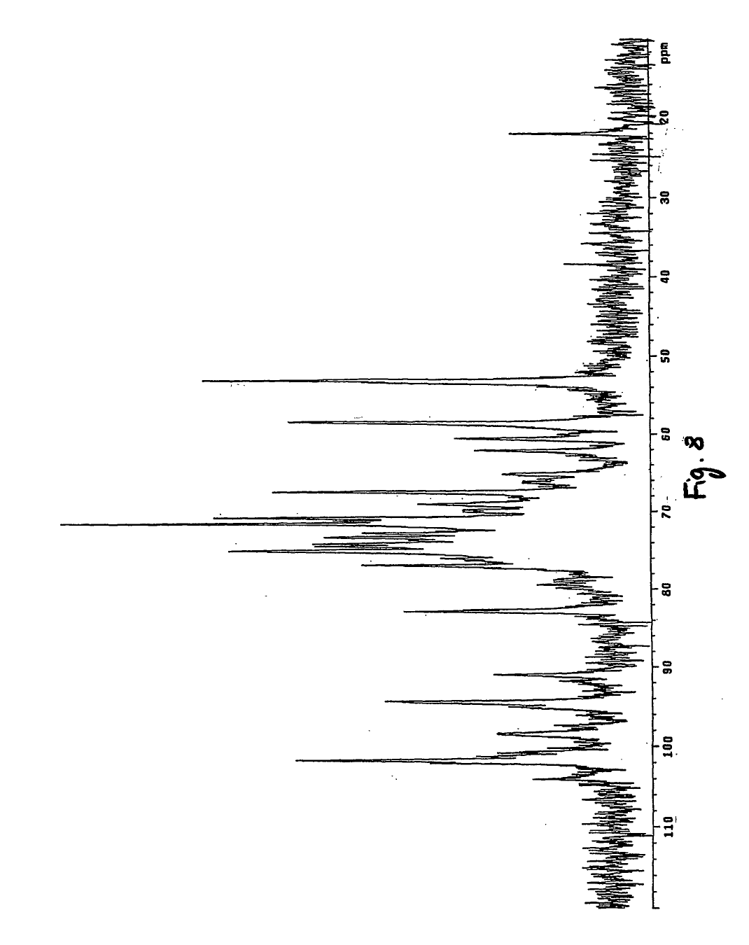

The ¹³C-NMR analysis on a freeze-dried aliquot of the supersulfated product is shown in FIG. 7.

e) The solution containing the product of the step d) is passed on IR-120 H+ (50 ml) cationic exchange resin. After the passage of the solution the resin is washed with 3 volumes of deionized water. The permeate pH turns out to be greater than 6. The acid solution is taken to neutrality with pyridine. The resulting solution is concentrated to 1/10 of the volume in a rotating evaporator at 40° C. under vacuum, and freeze-dried.

The obtained product, in form of pyridine salt, is added with 500 ml of a DMSO/methanol (9/1 VN) solution. The solution is kept at 60° C. for 3.5 hours and then it is added with 50 mi of deionized water and finally it is treated with 1,650 ml of acetone saturated with sodium chloride.

The obtained solid is purified by diafiltration as described in the step b) obtaining a solution with 10% concentration.

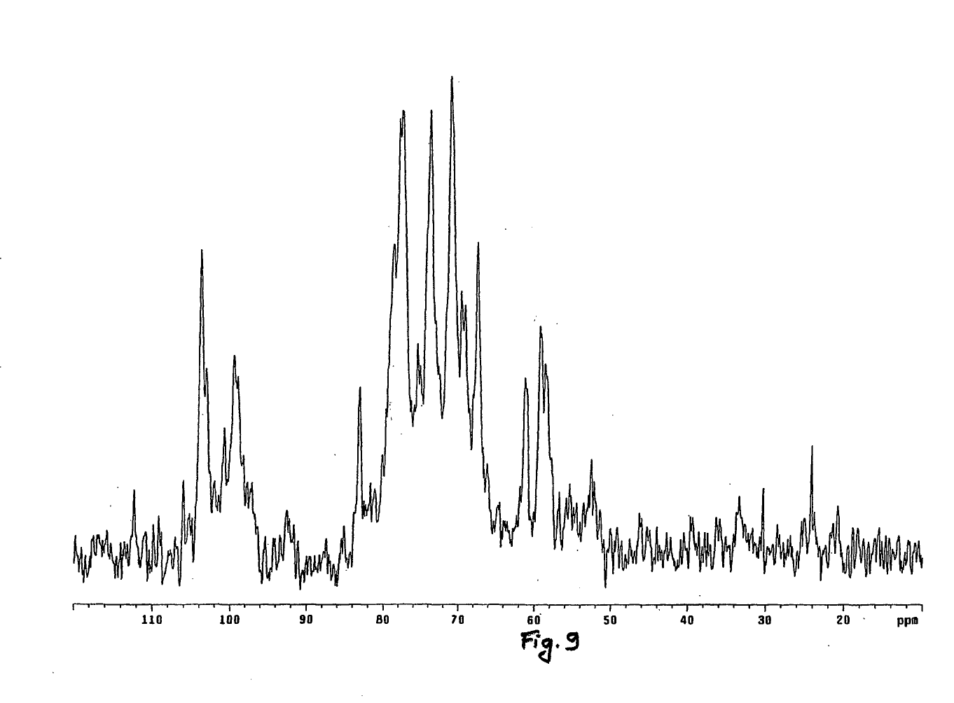

The ¹³C-NMR analysis on a freeze-dried aliquot is reported in FIG. 8 and it shows a sulfates in position 6 content of the aminosugar equal to 35%.

f) The solution containing the product of the step e) is passed on IR-120 H+ (50 mi) cationic exchange resin. After the passage of the solution the resin is washed with 3 volumes of deionized water. The permeate pH turns out to be greater than 6. The acid solution is taken to neutrality with a 15% tetrabutylammonium hydroxide aqueous solution. The resulting solution is concentrated to 1/10 of the volume in a rotating evaporator at 40° C. under vacuum, and freeze-dried.

The product, in form of tetrabutylammonium salt, is suspended in 200 ml of DMF.

The suspension is cooled to 0° C. and treated with 40 g of the pyridine-SO₃ adduct dissolved in 100 ml of DMF. The sulfating agent is added by single addition.

The solution is left at 0° C. for 1.5 hours and then it is treated with 750 ml of acetone saturated with sodium chloride.

The obtained solid is purified by diafiltration as described in the step b).

g) The solution coming from the step f) is treated as described in the step b) for the N-sulfation.

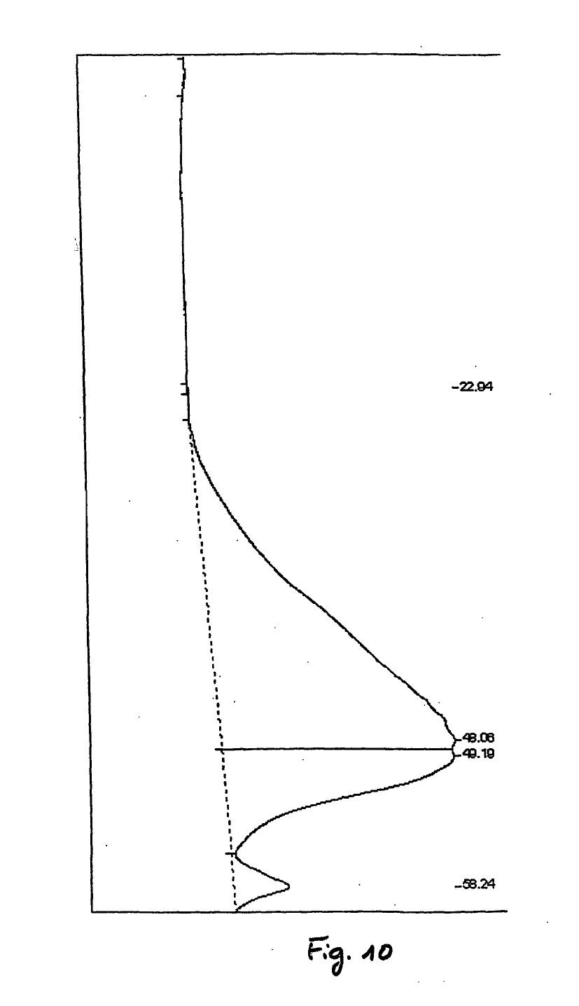

The ¹³C-NMR analysis on a freeze-dried aliquot of the obtained product is shown in FIG. 9.

The obtained product shows the chemico-physical and biological characteristics reported in Table 2-row 3 compared with the IV heparin international standard and with the I low molecular weight heparin international standard.

### EXAMPLE 2

The Example 1 has been repeated with the difference that in the step c) the immobilized C-5 epimerase enzyme has been used extracted from murine mastocytoma as described by Jacobsson et al., J. Biol. Chem. 254,2975-2982 (1979), with a reaction buffer containing 40 mM Caca2, pH 7.4.

The obtained product shows an iduronic acid/glucuronic acid ratio of 59.5:40.5 and the characteristics described in Table 2 row 4.

### EXAMPLE 3

The Example 1 has been repeated with the difference that in the step c) the immobilized C-5 epimerase enzyme has been used extracted from cattle-liver as described in W096/14425, with a reaction buffer at pH 7.4 and a reaction time equal to 32 hours.

Moreover in the step e) the reaction time has been 4 hours.

The obtained product shows an iduronic acid/glucuronic acid ratio of 55.4:44.6 and the characteristics described in Table 2 row 5.

### EXAMPLE 4

The Example 1 is repeated with the difference that in the step c) the recombinant C-5 epimerase enzyme in solution is used, using for the epimerization 10 g of N-deacetilated N-sulfate K5 dissolved in 1,000 ml of 25 mM HEPES buffer, pH 6.5, containing 50 mM CaCl2. To this solution 1.5×10¹¹ cpm equivalents of recombinant enzyme described in the Example 1 are added. The solution is kept at 37° C. for 24 hours. The solution is then treated at 100° C. for 10 minutes in order to denaturate the enzyme and finally it is filtered on 0.45μ filter to obtain the clear solution containing the product. The obtained product is then purified by diafiltration and precipitation with ethanol or acetone. The precipitate is resolubilized in water at a concentration equal to 10% and treated as in the Example 1 keeping however the reaction time of the step e) for 2 hours.

The obtained product shows an iduronic acid/glucuronic acid ratio of 56:44 and the characteristics described in Table 2 row 6.

### EXAMPLE 5

The Example 4 is repeated using in the step c) the enzyme from murine mastocytoma already described in the Example 2, in solution, with reaction buffer at pH 7.4 containing 40 mM BaCl₂ and maintaining the reaction for 18 hours.

Moreover in the step e) the reaction time is 3 hours. The obtained product shows an iduronic acid/glucuronic acid ratio of 40.1:59.9 and the characteristics described in Table 2 row 7.

### EXAMPLE 6

The Example 4 is repeated using in the step c) the C-5 epimerase enzyme from cattle-liver already described in the Example 3, in solution with reaction buffer containing 12.5 mM MnCI2 and maintaining the reaction for 14 hours. Moreover in the step e) the reaction time is 4 hours. The obtained product shows a iduronic acid/glucuronic acid ratio of 44.3:55.7 and the characteristics described in Table 2 row 8.

### EXAMPLE 7

The Example 4 is repeated using in the step c) a reaction buffer at pH 7.4 containing 37.5 MM MgCl₂ and maintaining the reaction for 16 hours. Moreover in the step e) the reaction time is 4 hours.

The obtained product shows an iduronic acid/glucuronic acid ratio of 47.5:52.5 and the characteristics described in Table 2 row 9.

### EXAMPLE 8

The Example 3 is repeated using in the step c) a reaction buffer at pH 7.0 containing 10 mM MgCl₂, 5 mM CaCl₂, 10 mM MnCl₂ and maintaining the reaction for 24 hours.

Moreover in the step e) the reaction time is 3 hours.

The obtained product shows an iduronic acid/glucuronic acid ratio of 44.8:55.2 and the characteristics described in Table 2 row 10.

### EXAMPLE 9

The Example 6 is repeated using in the step c) a reaction buffer at pH 7.4 containing 10 mM MgCl₂, 5 mM CaCl₂, 10 mM MnCl₂ and maintaining the reaction for 24 hours.

Moreover in the step e) the reaction time is 3 hours.

The obtained product shows an iduronic acid/glucuronic acid ratio of 52:48 and the characteristics described in Table 2 row 11.

### EXAMPLE 10

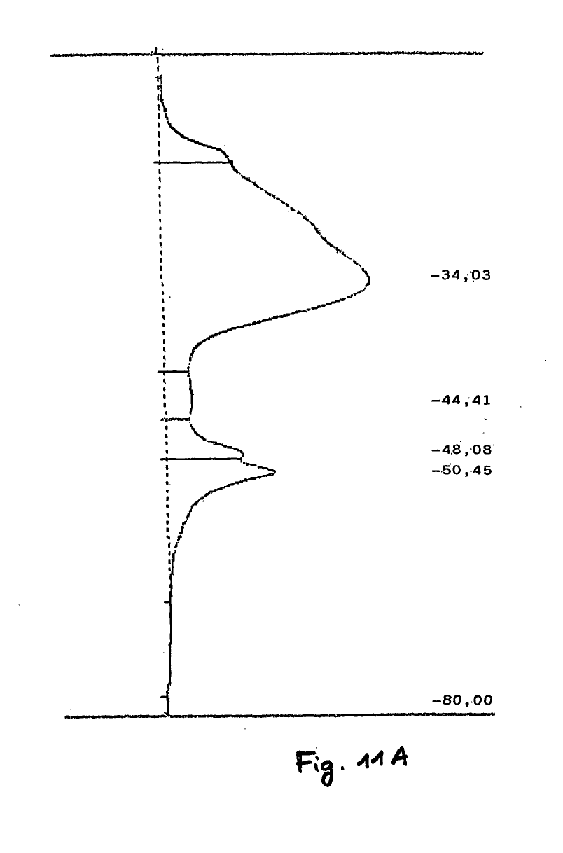

The sample obtained in the Example 3 having a molecular weight distribution obtained according Harenberg and De Vries, J. Chromatography 261, 287-292 (1983) (FIG. 10) is submitted to separation by gel filtration technique. In particular 1 gram of product is dissolved in 20 ml of 1 M NaCI buffer solution and deposed on a column containing 1,000 ml of Sephacryl HR S-400 (Amersham-Pharmacia) resin. The column is then eluted with 2,000 ml of 1 M NaCI buffer solution and gathered in 50 ml equal fractions by fraction collector (Gilson). After the determination of the product content on each fraction by carbazole analysis (Bitter and Muir, Anal. Biochem. 39,88-92-1971) the resulting fractions containing the sample are grouped in fraction A and fraction B respectively corresponding to the high molecular weight and low molecular weight portions. These fractions after concentration to 10 per cent of the volume by evaporator under vacuum are desalted in a column containing 500 ml of Sephadex G-10 (Amersham-Pharmacia) resin.

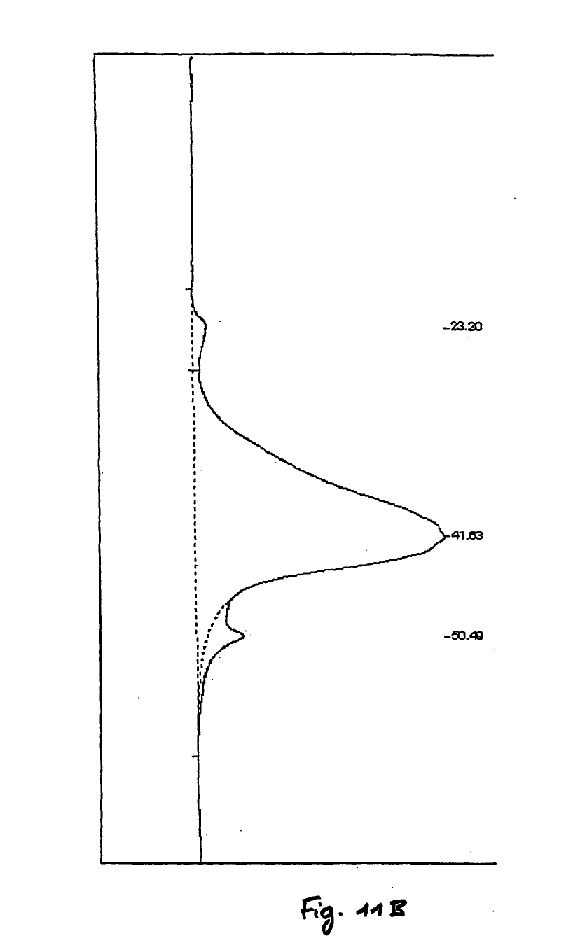

The solutions containing the desalted products are freeze-dried obtaining the fraction A and the fraction B (FIG. 11A and FIG. 11B). The obtained products show the characteristics described in Table 2 rows 12 and 13.

### EXAMPLE 11

The sample obtained in the Example 4 is submitted to controlled degradation with nitrous acid as described in the WO 8203627 patent. In particular 5 g of sample are dissolved in 250 ml of water and taken to 4° C. with thermostated bath. The pH is taken to 2.0 with 1 N hydrochloric acid cooled to 4° C. and then 10 ml of a 1% sodium nitrite solution are quickly added. If necessary the pH is taken back to 2 with 1 N hydrochloric acid and it is kept under slow stirring for 15 minutes. The solution is neutralized with 1N NaOH cooled to 4° C. Then 250 mg of sodium boron hydride dissolved in 13 ml of deionized water are added and it is left to react for 4 hours. It is taken to pH 5.0 with 1 N hydrochloric acid and it is left for 10 minutes in order to destroy the sodium boron hydride excess, and then it is neutralized with 1 N NaOH. The product is recovered by precipitation with 3 volumes of ethanol and then it is dried in vacuum stove. The obtained product shows the characteristics described in Table 2 row 14.

TABLE 2

| Anticoagulant | | | | | | |
|---|---|---|---|---|---|---|
| and | | | | | | |
| antithrombotic | | | | | | |
| activity of the | | | | | | 6) |
| products from | 1) Anti | 2) | 3) | 4) Anti | | ATIII |
| the described | Xa | APTT | HCII | IIa | | Affinity |
| examples | (%) | (%) | (%) | (%) | 5) MW | (%) |
| UF Heparin | 100 | 100 | 100 | 100 | 13500 | 32% |
| (4^{th} Int. STD) | | | | | | |
| LMW | 84 | 30 | | 33 | 4500 | n.d. |
| Heparin | | | | | | |
| (1^{st} Int. Std) | | | | | | |
| Example 1 | 76.6 | 43.4 | 256 | 118 | 15200 | 29 |
| Example 2 | 94.3 | 57 | 294 | 208 | 13500 | 29.5 |
| Example 3 | 112 | 88 | 346 | 223 | 14600 | 28 |
| Example 4 | 157 | 71.5 | 362 | 600 | 22500 | 29 |
| Example 5 | 150 | 70 | 352 | 213 | 24000 | 31 |
| Example 6 | 150 | 79 | 335 | 333 | 23000 | 33 |
| Example 7 | 120 | 92 | 346 | 247 | 13000 | 29 |
| Example 8 | 153 | 75 | 332 | 240 | 22500 | 34 |
| Example 9 | 157 | 71 | 346 | 233 | 23000 | 35 |
| Example 10-A | 250 | 70.8 | 480 | 435 | 30000 | 48 |
| Example 10-B | 43 | 77.7 | 145 | 27.3 | 7600 | 24 |
| Example 11 | 97.5 | 55.5 | 230 | 210 | 5400 | 25 |

The references from 1) to 6) have the meaning described for Table 1.

From the Table one points out that the product obtained with the present process shows activities comparable with the extractive heparin in the tests relating to the Xa factor (1) while the global (2) anticoagulant activity is reduced and those tests referring to the inhibition of thrombin (3,4) are significantly greater. These characteristics configure in the product greater antithrombotic properties and less side effects such as the bleeding effect with respect to the extractive heparin.

1-6. (canceled)

7. A process for the preparation of N-deacetylated N-sulfated derivatives of the K5 polysaccharide, epimerized at least to 40% of L-iduronic acid with respect to the total of uronic acids, having a molecular weight from 2,000 to 30,000 D, containing from 25 to 50%, by weight, of the chains having high affinity for ATIII and having an anticoagulant and antithrombotic activity expressed as HCII/antiXa ratio ranging from 1.5 to 4 comprising in sequence the preparation of the K5 polysaccharide from Escherichia Coli, N-deacetylation, and N-sulfation, C-5 epimerization of the D-glucuronic acid to the L-iduronic acid, supersulfation, selective O-desulfation, selective 6-O-sulfation and N-sulfation, characterized in that said C-5 epimerization is carried out by the use of the glucoronsyl C-5 epimerase enzyme:
(i) in solution by dissolution of an amount of the C-5 epimerase enzyme ranging from 1.2×10⁷ to 1.2×10¹¹ cpm in 2 to 2,000 ml of 25 mM Hepes buffer at a pH from 5.5 to 7.4 containing from 0.001 to 10 g of N-deacetylated N-sulfated K5 and one or a combination of said cations at a concentration ranging from 10 to 60 mM or
(ii) in immobilized form in the presence of divalent cations by recirculation of 20 to 1,000 ml of a 25 mM Hepes buffer solution at a pH from 6 to 7.4, containing 0.001 to 10 g of N-deacetylated N-sulfated K5 and one of said cations at a concentration ranging 10 to 60 mM, through a column containing from 1.2×10⁷ to 3×10¹¹ cpm of the enzyme immobilized on an inert support.

8-10. (canceled)

11. A pharmaceutical composition having anticoagulant and antithrombotic properties for the treatment of mammals, said composition containing an effective amount of one or more N-deacetylated N-sulfated derivatives of the k5 polysaccharide, epimerized at least to 40% of L-iduronic acid with respect to the total of uronic acids, having molecular weight from 2,000 to 30,000 D, containing from 25 to 50% by weight of the chains having high affinity for ATIII and having an anticoagulant and antithrombotic activity expressed as HCII/antiXa ratio ranging from 1.5 to 4 in combination with pharmaceutically acceptable excipients or diluents.

12. A therapeutic method for the anticoagulant and antithrombotic treatment of the blood of a mammal requiring such therapy, said method consisting of the administration of from 1 to 100 mg/day of one or more N-deacetylated N-sulfated derivatives of the k5 polysaccharide, epimerized at least to 40% of L-iduronic acid with respect to the total of uronic acids, having molecular weight from 2,000 to 30,000 D, containing from 25 to 50% by weight of the chains having high affinity for ATIII and having an anticoagulant and antithrombotic activity expressed as HCII/antiXa ratio ranging from 1.5 to 4.

13. A process according to claim 7, wherein said selective O-desulfation step is carried out by reacting a tertiary amine or quaternary ammonium salt of the oversulfated product with a solution of dimethyl sulfoxide/methanol 9/1 (V/V) at 60° C. for 3 hours.

14. A process according to claim 7, wherein said C5 epimerization step is performed using the enzyme glucuronosyl C5 epimerase in solution or in immobilized form in the presence of divalent cations, said selective O-desulfation step is carried out by reacting a tertiary or quaternary ammonium salt of the oversulfated product with a solution of dimethyl sulfoxide/methanol 9/1 (V/V) at 60° C. for 3 hours and said selective step is performed by reacting a tertiary amine or quaternary ammonium salt of the selectivity O-desulfated product with a calculated amount of a sulfating agent at a temperature of 0-5° C. for 0.5-3 hours.

15. A process according to claim 14 wherein said selective 6-O-sulfation step is carried out for 1.5 hours using a pyridine sulfur trioxide adduct as sulfating agent.

16. A process for the preparation of K5 glycosaminoglycans comprising the steps of (i) N-deacetylation/N-sulfation of the polysaccharide K5, (ii) partial C-5-epimerization of the carboxyl group of the glucuronic acid moiety to the corresponding iduronic acid moiety, (iii) oversulfation, (iv) selective O-desulfation, (v) optional 6-O-sulfation, and (vi) N-sulfation, in which step (iv) comprises treating the oversulfated product obtained at the end of step (iii) with a mixture methanol/dimethyl sulfoxide for a period of time from 135 to 165 minutes.

17. A process according to claim 16 in which said period of time is of about 150 minutes.

18. A process according to claim 16 in which said treatment is made for a period of time of about 150 minutes at a temperature of about 60° C.

19. A process for the preparation of novel glycosaminoglycans, which comprises:
(i) reacting polysaccharide K5 with a N-deacetylating agent, then treating the N-deacetylated product with a N-sulfating agent;
(ii) submitting the N-sulfate K5 thus obtained to a C5-epimerization by glucuronosyl C5 epimerase to obtain a C5-epimerized N-sulfate K5 in which the iduronic/glucuronic ratio is from 60/40 to 40/60;
(iii) converting the C5 epimerized N-sulfate K5, having a content of 40 to 60% iduronic acid over the total uronic acids, into a tertiary amine or quaternary ammonium salt thereof, then treating the salt thus obtained with an O-sulfating agent in a aprotic polar solvent at a temperature of 40-60° C. for 10-20 hours;
(iv) treating an organic base salt of the O-oversulfated product thus obtained with a mixture of dimethyl sulfoxide/methanol at 50-70° C. for 135-165 minutes to perform a partial O-desulfation;
(v) treating an organic base salt of the partially O-desulfated product thus obtained with an O-sulfating agent at a temperature of 0-5° C. to perform a 6-O-sulfation.
(vi) treating the O-sulfated product thus obtained with a N-sulfating agent;
whatever product obtained at the end of one of steps (ii) to (vi) being optionally submitted to a depolymerization.

20. A process according to claim 19, wherein a previously purified K5 is used as starting material.

21. A process according to claim 19, wherein, in step (i), hydrazine or a salt thereof or an alkaline metal hydroxide is used as a N-deacetylating agent and pyridine sulfur trioxide or trimethylamine sulfur trioxide adduct is used as a N-sulfating agent.

22. A process according to claim 19 wherein, in step (ii), said C5 epimerization is performed using the enzyme glucuronosyl C5 epimerase in solution or in immobilized form in presence of divalent cations.

23. A process according to claim 22, wherein said divalent cations comprise at least one of Ba, Ca, Mg and Mn.

24. A process according to claim 19, wherein, in step (ii), said epimerase is selected from the group consisting of recombinant glucuronosyl C5 epimerase, glucuronosyl C5 epimerase from murine mastocytoma and glucuronosyl C5 epimerase extracted from bovine liver.

25. A process according to claim 22, wherein said C5 epimerization with the enzyme in this immobilized form is performed and comprises recirculating 20-1,000 ml of a solution of 25 mM Hepes at pH of from 6 to 7.4 containing 0.001-10 g of N-deacetylated N-sulfated K5 and one of said cations at a concentration between 10 and 60 mM through a column containing from 1.2×10⁷ to 3×10¹¹ cpm of the immobilized enzyme on an inert support.

26. A process according to claim 25, wherein said pH is of about 7 and said C5 epimerization is performed with a recombinant enzyme at a temperature of about 30° C. by recirculating said solution with a flow rate of from 30 to 220 ml/hour for a time of about 24 hours.

27. A process according to claim 19, wherein, in step (iii), the pyridine sulfur trioxide adduct is used as O-sulfating agent.

28. A process according to claim 19, wherein, in step (iv), the reaction is carried out in dimethyl sulfoxide/methanol 9/1 (V/V) at about 60° C. for about 150 minutes.

29. A process according to claim 19, wherein a previously purified K5 is used as starting material and, in step (iv), the reaction is carried out in dimethyl sulfoxide/methanol 9/1 (V/V) at about 60° C. for about 150 minutes.

30. A process according to claim 19, wherein, in step (v), the 6-O-sulfation is carried out at 0.5° C. by using the pyridine sulfur trioxide adduct as O-sulfating agent.

31. A process according to claim 19, wherein, in step (vi), pyridine.sulfur trioxide or trimethylamine.sulfur trioxide adduct is used as N-sulfating agent.

32. A process according to claim 19, wherein the product obtained at the end of step (vi) is submitted to a nitrous acid depolymerization followed by a reduction by sodium borohydride.

33. A process according to claim 19, wherein a previously purified K5 is used as starting material and, in step (iv), the reaction is carried out in dimethyl sulfoxide/methanol 9/1 (V/V) at about 60° C. for about 150 minutes, and the C5-epimerized N,O-sulfate K5 glycosaminoglycan obtained at the end of step (vi) is submitted to a nitrous acid depolymerization followed by a reduction by sodium borohydride.

34. A process according to claim 19, wherein the glycosaminoglycan thus obtained is isolated in form of its sodium salt.

35. A process according to claim 34, wherein said sodium salt is further converted into another salt of said glycosaminoglycan.

36. A process according to claim 35, wherein said other salt is another alkaline metal, or an alkaline-earth metal, ammonium, tetra(C₁-C₄)alkylammonium, aluminum or zinc salt.

37. A glycosaminoglycan constituted by a mixture of chains in which at least 90% of said chains has the formula I 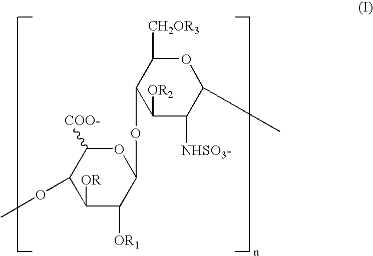
wherein 40-60% of the uronic acid units are those of iduronic acid, n is an integer from 3 to 100, R, R₁, R₂ and R₃ represent a hydrogen atom or a SO₃⁻ group and from about 65% to about 50% of R, R₁, R₂ and R₃ being hydrogen and the remaining being SO₃⁻ groups distributed as follows
R₃ is from about 85% to about 95% SO₃⁻;
R₂ is from about 17 to about 21% SO₃⁻;
R₁ is from about 15 to about 35% SO₃⁻ in iduronic units and 0 to 5% SO₃⁻ in glucuronic units;
R is from about 20 to about 40% SO₃⁻ in glucuronic units and 0 to 5% in iduronic units;
the sum of the SO₃⁻% in R₁ glucoronic units, and in R, iduronic units, is from 3 to 7%;
R₁ and R being not simultaneously SO₃⁻ and being both hydrogen in 25-45% of the uronic acid units; the sulfation degree being from about 2.3 to about 2.9, and the corresponding cation being a chemically or pharmaceutically acceptable one.

38. The glycosaminoglycan of claim 37, wherein said corresponding cation is an alkaline metal, alkaline-earth metal, aluminum or zinc ion.

39. The glycosaminoglycan of claim 37, wherein said corresponding cation is sodium or calcium ion.

40. The glycosaminoglycan of claim 37, wherein from about 60% to about 55% of R, R₁, R₂ and R₃, taken together, are hydrogen and the remaining are SO₃⁻ groups for a sulfation degree of from about 2.4 to about 2.7.

41. The glycosaminoglycan of claim 37, wherein at least 80% of said chains in said mixture of chains have the formula I wherein n is from 3 to 15.

42. The glycosaminoglycan of claim 41, wherein said chains in said mixture of chains has a molecular weight distribution ranging from about 2,000 to about 10,000, with a mean molecular weight of from about 4,000 to about 8,000.

43. The glycosaminoglycan of claim 42 wherein said chains in said mixture of chains have a mean molecular weight of about 7,000 and at least 90% of said mixture of chains has the formula I, 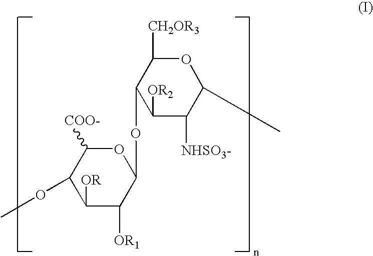
wherein about 55% of the uronic acid units are those of iduronic acid and
R₃ is about 85% SO₃⁻;
R₂ is about 20% SO₃⁻;
R₁ is about 25% SO₃⁻ in iduronic units and 0 to about 5% SO₃⁻ in glucuronic units;
R is about 30% SO₃⁻ in glucuronic units and 0 to about 5% in iduronic units;
the sum of the SO₃⁻ percent in R₁, glucuronic units and in R, iduronic units, is about 5%;
R₁ and R being not simultaneously SO₃⁻ and being both hydrogen in about 40% of the uronic acid units; the sulfation degree being about 2.55, the corresponding cation being a chemically or pharmaceutically acceptable one.

44. The glycosaminoglycan of claim 43, wherein said corresponding cation is an alkaline metal, alkaline-earth metal, aluminum or zinc ion.

45. The glycosaminoglycan of claim 43, wherein said corresponding cation is sodium or calcium ion.

46. The glycosaminoglycan of claim 43, wherein said mixture of chains has a mean molecular weight of 7,400.

47. The glycosaminoglycan of claim 37, wherein at least 80% of said chains in said mixture of chains have the structure I wherein n is from 20 to 100.

48. The glycosaminoglycan of claim 47 wherein said mixture of chains has a molecular weight distribution ranging from about 9,000 to about 60,000, with a mean molecular weight of from about 12,000 to about 30,000.

49. The glycosaminoglycan of claim 48, wherein said chains in said mixture of chains have a mean molecular weight of 14,000-16,000 and at least 90% of said chains have the formula I 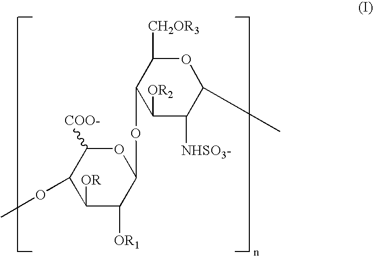
wherein about 55% of the uronic acid units are those of iduronic acid and
R₃ is from about 85% to about 90% SO₃⁻;
R₂ is about 20% SO₃⁻;
R₁ is from about 25% to about 30 SO₃⁻ in iduronic units and 0 to about 5% SO₃⁻ in glucuronic units;
R is from about 30% to about 35% SO₃⁻ in glucuronic units and 0 to about 5% in iduronic units;
the sum of the SO₃⁻% in R₁, glucuronic units and in R, iduronic units, is about 5%; R₁ and R being not simultaneously SO₃⁻ and being both hydrogen in from about 30% to about 40% of the uronic acid units; the sulfation degree being from about 2.5 to about 2.7, the corresponding cation being a chemically or pharmaceutically acceptable one.

50. The glycosaminoglycans of claim 49, wherein said corresponding cation is an alkaline metal, alkaline-earth metal, aluminum or zinc ion.

51. The glycosaminoglycan of claim 49 wherein said corresponding cation is sodium or calcium ion.

52. The glycosaminoglycan of claim 49, wherein said mixture of chains has a mean molecular weight of 15,700.

53. A pharmaceutical composition comprising a pharmacologically effective amount of a glycosaminoglycan constituted by a mixture of chains in which at least 90% of said chains has the formula I 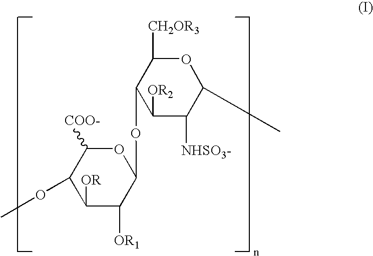
wherein 40-60% of the uronic acid units are those of iduronic acid, n is an integer from 3 to 100, R, R₁, R₂ and R₃ represent a hydrogen atom or a SO₃⁻ group and from about 65% to about 50% of R, R₁, R₂ and R₃ being hydrogen and the remaining being SO₃⁻ groups distributed as follows
R₃ is from about 85% to about 95% SO₃⁻;
R₂ is from about 17 to about 21% SO₃⁻;
R₁ is from about 15 to about 35% SO₃⁻ in iduronic units and 0 to 5% SO₃⁻ in glucuronic units;
R is from about 20 to about 40% SO₃⁻ in gluc[o]uronic units and 0 to 5% in iduronic units;
the sum of the SO₃⁻ percent in R₁, glucuronic units, and in R, iduronic units, is from 3 to 7%;
R₁ and R being not simultaneously SO₃⁻ and being both hydrogen in 25-45% of the uronic acid units; the sulfation degree being from about 2.3 to about 2.9, and the corresponding cation being a pharmaceutically acceptable one, as an active ingredient, and a pharmaceutically acceptable carrier.

54. The composition of claim 53, wherein said glycosaminoglycan is constituted by a mixture of chains in which at least 80% of said chains have the formula I, in which n is from 3 to 15.

55. The composition of claim 54, wherein said mixture of chains has a molecular weight distribution ranging from about 2,000 to about 10,000 with a mean molecular weight of from about 4,000 to about 8,000.

56. The composition of claim 55, wherein said mixture of chains has a mean molecular weight of about 7,000 and at least 90% of said chains has the formula I 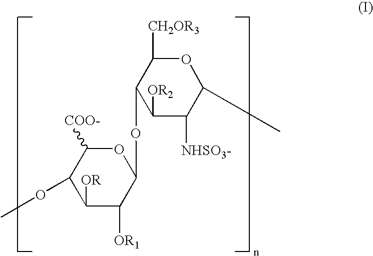
wherein about 55% of the uronic acid units are those of iduronic acid and
R₃ is about 85% SO₃⁻;
R₂ is about 20% SO₃⁻;
R₁ is about 25% SO₃⁻ in iduronic units and 0 to about 5% SO₃⁻ in glucuronic units;
R is about 30% SO₃⁻ in glucuronic units and 0 to about 5% in iduronic units;
the sum of the SO₃⁻ percent in R₁, glucuronic units, and in R, iduronic units, is about 5%;
R₁ and R being not simultaneously SO₃⁻ and being both hydrogen in about 40% of the uronic acid units; the sulfation degree being about 2.55, the corresponding cation being a pharmaceutically acceptable one.

57. The composition of claim 56, wherein said corresponding cation is an alkaline metal, alkaline-earth metal, aluminum or zinc ion.

58. The composition of claim 57, wherein said corresponding cation is sodium or calcium ion.

59. The composition of claim 58, wherein said mixture of chains has a mean molecular weight of 7,400.

60. A method for lessening coagulation in a mammal, which comprises administering to said mammal, in need of said coagulation lessening, a pharmacologically effective amount of the C5-epimerized N,O-sulfate K5 glycosaminoglycan of claim 37.

61. A method for lessening coagulation in a mammal, which comprises administering to said mammal, in need of said coagulation lessening, a pharmacologically effective amount of the glycosaminoglycan of claim 37.

62. A method for treating thrombosis in a mammal which comprises administering to said mammal an effective amount of the C5-epimerized N,O-sulfate K5 glycosaminoglycan of claim 37.

63. A method for treating thrombosis in a mammal which comprises administering to said mammal an effective amount of the glycosaminoglycan of claim 37.

64. The method of claim 60, wherein said effective amount is administered in a pharmaceutical composition containing from 5 to 100 mg of said glycosaminoglycan.

65. The method of claim 61, wherein said effective amount is administered in a pharmaceutical composition containing from 5 to 100 mg of said glycosaminoglycan.

66. The method of claim 62, wherein said effective amount is administered in a pharmaceutical composition containing from 5 to 100 mg. of said glycosaminoglycan.

67. The method of claim 63, wherein said effective amount is administered in a pharmaceutical composition containing from 5 to 100 mg of said glycosaminoglycan.

68. A process for the preparation of N-deacetylate N-sulfate derivatives of K5 polysaccharide, epimerized at least till 40% of iduronic acid with respect to the total uronic acids, having molecular weight from 2,000 to 30,000 D, containing from 25 to 50% in weight of the chains with high affinity for ATIII and having an anticoagulant and antithrombotic activity expressed as ratio HCII/Anti-Xa comprised between 1.5 and 4, said process comprising in sequence (a) the preparation of K5 polysaccharide from Escherichia coli, (b) N-deacetylation and N-sulfation, (c) C5 epimerization of D-glucuronic acid to L-iduronic acid, (d) oversulfation, (e) selective O-desulfation, (f) selective 6-O-sulfation and (g) N-sulfation, wherein
said C5 epimerization is performed using the enzyme glucuronosyl C5 epimerase in solution or in the presence of divalent cations;
said oversulfation of step (d) is performed by treating a tertiary amine or quaternary ammonium salt of the C5-epimerized product obtained at the end of step (c) with a sulfating agent at 20-70° for 2-24 hours to perform an O-oversulfation; and
said selective O-desulfation of step (e) is performed by treating the N-desulfated and O-oversulfated product obtained at the end of step (d) with a solution of dimethyl sulfoxide/methanol 9/1 (V/V) at 45-90° C. for 1-8 hours.

69. The process of claim 68, wherein in said step (d) said salt of said C5-epimerized product is the tetrabutylammonium salt.

70. The process of claim 69, wherein in said oversulfation step (d) said sulfating agent is pyridine.SO₃.

71. The process of claim 70, wherein said oversulfation is carried out in dimethyl formamide or dimethyl sulfoxide solution.

72. The process of claim 68, wherein said 6-O-sulfation of step (f) is performed by treating a tertiary amine or quaternary ammonium salt of the partially O-desulfated product obtained at the end of step (e) with a sulfating agent at 0-5° C. for 2-24 hours.

73. The process of claim 72, wherein in said step (f) said salt of the partially O-desulfated product is the tetrabutylammonium salt.

74. The process of claim 73, wherein said sulfation is carried out in dimethyl formamide or dimethyl sulfoxide solution.
